(19) United States
(12) Patent Application Publication
(10) Pub. No.: US 2020/0230085 A1
(43) Pub. Date: Jul. 23, 2020

(54) METHODS OF TREATING DISEASE WITH DICHLORPHENAMIDE

(71) Applicant: Strongbridge Dublin Limited, Trevose, PA (US)

(72) Inventor: Fredric Jay COHEN, Washington Crossing, PA (US)

(21) Appl. No.: 16/253,515

(22) Filed: Jan. 22, 2019

(51) Int. Cl. A61K 31/18; A61K 31/341; A61K 31/136; A61K 31/192; A61K 31/4184; A61K 31/407; A61B 5/00

(52) U.S. Cl. CPC A61K 31/18; A61K 31/341; A61K 31/136; A61B 5/4848; A61K 31/4184; A61K 31/407; A61K 31/192

(57) ABSTRACT

Provided herein is a method of administering dichlorphenamide, or a pharmaceutically acceptable salt thereof, to a subject in need thereof, wherein the subject is also being administered an organic anion transporter-1 (OAT1) inhibitor and/or an organic anion transporter-3 (OAT3) inhibitor. The method comprises administering to the subject a therapeutically effective amount of dichlorphenamide, or a pharmaceutically acceptable salt thereof, monitoring the subject for signs and/or symptoms of toxicity associated with the dichlorphenamide, or a pharmaceutically acceptable salt thereof, and adjusting the therapeutically effective amount of dichlorphenamide, or a pharmaceutically acceptable salt thereof, when the subject is experiencing a sign and/or symptom of toxicity associated with the dichlorphenamide, or a pharmaceutically acceptable salt thereof.

The present disclosure relates to new compositions, and their application as pharmaceuticals for treating disease. Methods of treating hyperkalemic periodic paralysis, hypokalemic periodic paralysis and other diseases in a human or animal subject are also provided.

Numerous endo- and xenobiotics including many drugs are organic anions or cations. Their disposition and elimination depend on the proper function of multispecific drug transporters that belong to two major superfamilies: solute carrier (SLC) transporters and ATP-binding cassette (ABC) transporters. Although most can transport bidirectionally, in general, ABC transporters are responsible for efflux of substrates, while SLC transporters mediate influx of substrates.

Dichlorphenamide (Keveyis®, Daranide™) is a carbonic anhydrase inhibitor approved for treating primary hyperkalemic periodic paralysis, primary hypokalemic periodic paralysis, and related variants, and has been used to treat elevated intraocular pressure (IOP). Dichlorphenamide was introduced by Merck in 1950's to treat glaucoma. Dichlorphenamide is now available as immediate-release tablets for oral administration, each containing 50 mg dichlorphenamide.

It has been discovered that, dichlorphenamide is a substrate of OAT1 and/or OAT3.

The present disclosure provides a method of administering dichlorphenamide, or a pharmaceutically acceptable salt thereof, to a subject in need thereof, wherein the subject is also being administered an organic anion transporter-1 (OAT1) inhibitor and/or an organic anion transporter-3 (OAT3) inhibitor. The method comprises administering to the subject a therapeutically effective amount of dichlorphenamide, or a pharmaceutically acceptable salt thereof, monitoring the subject for signs and/or symptoms of toxicity associated with the dichlorphenamide, or a pharmaceutically acceptable salt thereof, and adjusting the therapeutically effective amount of dichlorphenamide, or a pharmaceutically acceptable salt thereof, when the subject is experiencing a sign and/or symptom of toxicity associated with the dichlorphenamide, or a pharmaceutically acceptable salt thereof.

The present disclosure further provides a method of administering dichlorphenamide, or a pharmaceutically acceptable salt thereof, to a subject in need thereof. The method comprises administering to the subject a therapeutically effective amount of dichlorphenamide, or a pharmaceutically acceptable salt thereof, administering to the subject an organic anion transporter-1 (OAT1) inhibitor and/or an organic anion transporter-3 (OAT3) inhibitor, monitoring the subject for signs and/or symptoms of toxicity associated with the dichlorphenamide, or a pharmaceutically acceptable salt thereof, and adjusting the therapeutically effective amount of dichlorphenamide, or a pharmaceutically acceptable salt thereof, when the subject is experiencing a sign and/or symptom of toxicity associated with the dichlorphenamide, or a pharmaceutically acceptable salt thereof.

These and other aspects of the invention will be apparent upon reference to the following detailed description. To this end, various references are set forth herein which describe in more detail certain background information, procedures, compounds, and/or compositions, and are each hereby incorporated by reference in their entirety.

### DETAILED DESCRIPTION

Dichlorphenamide is a dichlorinated benzenedisulfonamide, known chemically as 4,5-dichloro-1,3-benzenedisulfonamide. Its empirical formula is C₆H₆Cl₂N₂O₄S₂ and its structural formula is:

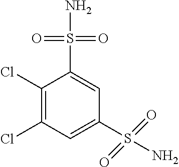 [SMILES: NS(=O)(=O)c1cc(Cl)c(Cl)c(S(N)(=O)=O)c1]

Dichlorphenamide USP is a white or practically white, crystalline compound with a molecular weight of 305.16 g/mol. It is very slightly soluble in water but soluble in dilute solutions of sodium carbonate and sodium hydroxide. Dilute alkaline solutions of dichlorphenamide are stable at room temperature. Dichlorphenamide is storage-stable for at least 36 months.

A formulation of dichlorphenamide has been previously reported in the United States Food and Drug Administration (FDA) approved drug label for Keveyis®, which is indicated for treating primary hyperkalemic periodic paralysis (“hyper PP”), primary hypokalemic periodic paralysis (“hypo PP”), and related variants, a heterogenous group of conditions for which responses may vary. The initial dose is 50 mg/day twice daily (bis in diem, BID), which may be adjusted at weekly intervals up to 200 mg/day.

Dichlorphenamide is a carbonic anhydrase inhibitor. The precise mechanism by which dichlorphenamide exerts its therapeutic effects in patients with periodic paralysis is unknown. It is hypothesized that dichlorphenamide modulates pH, which affects the resting membrane potential on muscle surfaces. In both hypo PP and hyper PP, skeletal muscle fibers intermittently become refractory to signals from motor neurons, leading to muscle weakness or flaccid paralysis.

When introducing elements of the present disclosure or the embodiment(s) thereof, the articles “a”, “an”, “the” and “said” are intended to mean that there are one or more of the elements. The terms “comprising”, “including” and “having” are inclusive and mean that there may be additional elements other than the listed elements.

The term “and/or” when in a list of two or more items, means that any of the listed items can be employed by itself or in combination with one or more of the listed items. For example, the expression “A and/or B” means either or both of A and B, i.e. A alone, B alone or A and B in combination. The expression “A, B and/or C” is intended to mean A alone, B alone, C alone, A and B in combination, A and C in combination, B and C in combination or A, B, and C in combination.

When ranges of values are disclosed, and the notation “from n₁ . . . to n₂” or “between n₁ . . . and n₂” is used, where n₁ and n₂ are the numbers, then unless otherwise specified, this notation is intended to include the numbers themselves and the range between them. This range may be integral or continuous between and including the end values. By way of example, the range “from 2 to 6 carbons” is intended to include two, three, four, five, and six carbons, since carbons come in integer units. Compare, by way of example, the range “from 1 to 3 μM (micromolar),” which is intended to include 1 μM, 3 μM, and everything in between to any number of significant figures (e.g., 1.255 μM, 2.1 μM, 2.9999 μM, etc.).

The term “about” qualifies the numerical values that it modifies, denoting such a value as variable within a margin of error. When no margin of error, such as a standard deviation to a mean value given in a chart or table of data, is recited, the term “about” means that range which would encompass the recited value and the range which would be included by rounding up or down to that figure, considering significant figures.

Any definition herein may be used in combination with any other definition to describe a composite structural group. By convention, the trailing element of any such definition is that which attaches to the parent moiety. For example, the composite group alkylamido would represent an alkyl group attached to the parent molecule through an amido group, and the term alkoxyalkyl would represent an alkoxy group attached to the parent molecule through an alkyl group.

The term “disease” as used herein is intended to be generally synonymous, and is used interchangeably with, the terms “disorder,” “syndrome,” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

The term “combination therapy” means the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment regimen will provide beneficial effects of the drug combination in treating the conditions or disorders described herein.

The phrase “therapeutically effective” is intended to qualify the amount of active ingredients used in the treating a disease or disorder or on the effecting of a clinical endpoint.

The term “therapeutically acceptable” refers to those compounds (or salts, prodrugs, tautomers, zwitterionic forms, etc.) suitable for use in contact with the tissues of patients without undue toxicity, irritation, and allergic response, are commensurate with a reasonable benefit/risk ratio, and are effective for their intended use.

As used herein, reference to “treatment” of a patient is intended to include prophylaxis. Treatment may also be preemptive in nature, i.e., it may include prevention of disease. Prevention of a disease may involve complete protection from disease, for example as in the case of prevention of infection with a pathogen or may involve prevention of disease progression. For example, prevention of a disease may not mean complete foreclosure of any effect related to the diseases at any level, but instead may mean prevention of the symptoms of a disease to a clinically significant or detectable level. Prevention of diseases may also mean prevention of progression of a disease to a later stage of the disease. In certain embodiments, prevention of a disease may involve prevention of attacks of an intermittent nature, as well as prevention of a permanent state of muscle weakness, such as an irreversible state of impairment owing to underlying disease.

The term “patient” is generally synonymous with the term “subject” and includes all mammals including humans. Examples of patients include humans, livestock such as cows, goats, sheep, pigs, and rabbits, and companion animals such as dogs, cats, rabbits, and horses. Preferably, the patient is a human.

As used herein, a patient is said to “tolerate” a dose of a compound if administering that dose to that patient does not result in an unacceptable adverse event or an unacceptable combination of adverse events. One of skill in the art will appreciate that tolerance is a subjective measure and that what may be tolerable to one patient may not be tolerable to a different patient. For example, one patient may not be able to tolerate headache, whereas a second patient may find headache tolerable but is not able to tolerate vomiting, whereas for a third patient, either headache alone or vomiting alone is tolerable, but the patient is not able to tolerate the combination of headache and vomiting, even if the severity of each is less than when experienced alone.

As used herein, an “adverse event” is an untoward medical occurrence associated with treatment with a pharmaceutical agent.

As used herein, “up-titration” of a compound refers to increasing the amount of a compound to achieve a therapeutic effect that occurs before dose-limiting intolerability for the patient. Up-titration can be achieved in one or more dose increments, which may be the same or different.

The term “prodrug” refers to a compound that is made more active in vivo. Certain compounds disclosed herein may also exist as prodrugs. Prodrugs of the compounds described herein are structurally modified forms of the compound that readily undergo chemical changes under physiological conditions to provide the compound. Additionally, prodrugs can be converted to the compound by chemical or biochemical methods in an ex vivo environment. For example, prodrugs can be slowly converted to a compound when placed in a transdermal patch reservoir with a suitable enzyme or chemical reagent. Prodrugs are often useful because, in some situations, they may be easier to administer than the compound, or parent drug. They may, for instance, be bioavailable by oral administration whereas the parent drug is not. The prodrug may also have improved solubility in pharmaceutical compositions over the parent drug. A wide variety of prodrug derivatives are known in the art, such as those that rely on hydrolytic cleavage or oxidative activation of the prodrug. An example, without limitation, of a prodrug would be a compound which is administered as an ester (the “prodrug”), but then is metabolically hydrolyzed to the carboxylic acid, the active entity. Additional examples include peptidyl derivatives of a compound.

The compounds disclosed herein can exist as therapeutically acceptable salts. The present disclosure includes compounds listed above in the form of salts, including acid addition salts. Suitable salts include those formed with both organic and inorganic acids. Such acid addition salts will normally be pharmaceutically acceptable. However, salts of non-pharmaceutically acceptable salts may be of utility in the preparation and purification of the compound in question. Basic addition salts may also be formed and be pharmaceutically acceptable.

The term “therapeutically acceptable salt,” as used herein, represents salts or zwitterionic forms of the compounds disclosed herein which are water or oil-soluble or dispersible and therapeutically acceptable as defined herein. The salts can be prepared during the final isolation and purification of the compounds or separately by reacting the appropriate compound in the form of the free base with a suitable acid. Representative acid addition salts include acetate, adipate, alginate, L-ascorbate, aspartate, benzoate, benzenesulfonate (besylate), bisulfate, butyrate, camphorate, camphorsulfonate, citrate, digluconate, formate, fumarate, gentisate, glutarate, glycerophosphate, glycolate, hemisulfate, heptanoate, hexanoate, hippurate, hydrochloride, hydrobromide, hydroiodide, 2-hydroxyethansulfonate (isethionate), lactate, maleate, malonate, DL-mandelate, mesitylenesulfonate, methanesulfonate, naphthylenesulfonate, nicotinate, 2-naphthalenesulfonate, oxalate, pamoate, pectinate, persulfate, 3-phenylproprionate, phosphonate, picrate, pivalate, propionate, pyroglutamate, succinate, sulfonate, tartrate, L-tartrate, trichloroacetate, trifluoroacetate, phosphate, glutamate, bicarbonate, para-toluenesulfonate (p-tosylate), and undecanoate. Also, basic groups in the compounds disclosed herein can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form therapeutically acceptable addition salts include inorganic acids such as hydrochloric, hydrobromic, sulfuric, and phosphoric, and organic acids such as oxalic, maleic, succinic, and citric. Salts can also be formed by coordination of the compounds with an alkali metal or alkaline earth ion. Hence, the present disclosure contemplates sodium, potassium, magnesium, and calcium salts of the compounds disclosed herein, and the like.

Basic addition salts can be prepared during the final isolation and purification of the compounds by reacting a carboxy group with a suitable base such as the hydroxide, carbonate, or bicarbonate of a metal cation or with ammonia or an organic primary, secondary, or tertiary amine. The cations of therapeutically acceptable salts include lithium, sodium, potassium, calcium, magnesium, and aluminum, as well as nontoxic quaternary amine cations such as ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine, and N,N′-dibenzylethylenediamine. Other representative organic amines useful for the formation of base addition salts include ethylenediamine, ethanolamine, diethanolamine, piperidine, and piperazine.

A salt of a compound can be made by reacting the appropriate compound in the form of the free base with the appropriate acid.

While the disclosed compounds may be administered as the raw chemical, it is also possible to present them as a pharmaceutical formulation. Accordingly, provided herein are pharmaceutical formulations which comprise one or more of certain compounds disclosed herein, or one or more pharmaceutically acceptable salts, esters, prodrugs, amides, or solvates thereof, together with one or more pharmaceutically acceptable carriers thereof and optionally one or more other therapeutic ingredients. The carrier(s) must be “acceptable” in the sense of being compatible with the other ingredients of the formulation and not deleterious to the recipient thereof. Proper formulation is dependent upon the route of administration chosen. Any of the well-known techniques, carriers, and excipients may be used as suitable and as understood in the art. The pharmaceutical compositions disclosed herein may be manufactured in any manner known in the art, e.g., by means of conventional mixing, dissolving, granulating, dragee-making, levigating, emulsifying, encapsulating, entrapping or compression processes.

The formulations include those suitable for oral, parenteral (including subcutaneous, intradermal, intramuscular, intravenous, intraarticular, and intramedullary), intraperitoneal, transmucosal, transdermal, rectal and topical (including dermal, buccal, sublingual and intraocular) administration although the most suitable route may depend upon for example the condition and disorder of the recipient. The formulations may conveniently be presented in unit dosage form and may be prepared by any of the methods well known in the art of pharmacy. Typically, these methods include the step of bringing into association a compound disclosed herein or a pharmaceutically acceptable salt, ester, amide, prodrug or solvate thereof (“active ingredient”) with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation.

Formulations of the compounds disclosed herein suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

Pharmaceutical preparations which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. The tablets may optionally be coated or scored and may be formulated to provide slow or controlled release of the active ingredient therein. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Dragee cores are provided with suitable coatings. For this purpose, concentrated sugar solutions may be used, which may optionally contain gum arabic, talc, polyvinyl pyrrolidone, Carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The formulations may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately before use. Extemporaneous injection solutions and suspensions may be prepared from sterile powders, granules and tablets of the kind previously described.

Formulations for parenteral administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

For buccal or sublingual administration, the compositions may take the form of tablets, lozenges, pastilles, or gels formulated in conventional manner. Such compositions may comprise the active ingredient in a flavored basis such as sucrose and acacia or tragacanth.

The compounds may also be formulated in rectal compositions such as suppositories or retention enemas, e.g., containing conventional suppository bases such as cocoa butter, polyethylene glycol, or other glycerides.

Certain compounds disclosed herein may be administered topically, that is by non-systemic administration. This includes the application of a compound disclosed herein externally to the epidermis or the buccal cavity and the instillation of such a compound into the ear, eye and nose, such that the compound does not significantly enter the blood stream. In contrast, systemic administration refers to oral, intravenous, intraperitoneal and intramuscular administration.

Formulations suitable for topical administration include liquid or semi-liquid preparations suitable for penetration through the skin to the site of inflammation such as gels, liniments, lotions, creams, ointments or pastes, and drops suitable for administration to the eye, ear or nose. The active ingredient for topical administration may comprise, for example, from 0.001% to 10% w/w (by weight) of the formulation. In certain embodiments, the active ingredient may comprise as much as 10% w/w. In other embodiments, it may comprise less than 5% w/w. In certain embodiments, the active ingredient may comprise from 2% w/w to 5% w/w. In other embodiments, it may comprise from 0.1% to 1% w/w of the formulation.

For administration by inhalation, compounds may be conveniently delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, the compounds may be a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator.

Preferred unit dosage formulations are those containing an effective dose, as herein below recited, or an appropriate fraction thereof, of the active ingredient.

In addition to the ingredients particularly mentioned above, the formulations described above may include other agents conventional in the art having regard to the type of formulation in question, for example those suitable for oral administration may include flavoring agents.

Compounds may be administered orally or via injection at a dose of from 0.1 to 500 mg/kg per day. The dose range for adult humans is generally from 5 mg to 2 g/day. Tablets or other forms of presentation provided in discrete units may conveniently contain an amount of one or more compounds which is effective at such dosage or as a multiple of the same, for instance, units containing 5 mg to 500 mg, usually around 10 mg to 200 mg.

In certain embodiments, the subject may receive a dose of between 50 mg twice daily and to 100 mg twice daily. In certain embodiments, the dose is 50 mg once daily. In certain embodiments, the dose is 50 mg once every other day. In certain embodiments, the dose is 25 mg once daily. In certain embodiments, the dose is 25 mg once every other day.

In certain embodiments, the therapeutically effective amount of the dichlorphenamide, or a pharmaceutically acceptable salt thereof, is between 25 mg and 200 mg per day.

In certain embodiments, the therapeutically effective amount of the dichlorphenamide, or a pharmaceutically acceptable salt thereof, is 50 mg twice daily.

In certain embodiments, the dichlorphenamide, or a pharmaceutically acceptable salt thereof, is administered via a titration scheme that comprises the up-titration of the dichlorphenamide, or a pharmaceutically acceptable salt thereof, at about weekly intervals until a modified dose is administered.

In certain embodiments, the modified dose of the dichlorphenamide, or a pharmaceutically acceptable salt thereof, is 200 mg. In certain embodiments, the modified dose of the dichlorphenamide, or a pharmaceutically acceptable salt thereof, is 150 mg. In certain embodiments, the modified dose of the dichlorphenamide, or a pharmaceutically acceptable salt thereof, is 100 mg.

In certain embodiments, dichlorphenamide, or a pharmaceutically acceptable salt thereof, is administered via a titration scheme that comprises administering a first dose of the of the dichlorphenamide, or a pharmaceutically acceptable salt thereof, for a period of one week; further increasing the dose by an amount equal to an incremental value; and determining whether the subject tolerates the further increased dose; wherein the cycle is repeated so long as the subject tolerates the further increased dose, wherein the incremental value at each cycle repetition is the same or different; and wherein if the subject does not tolerate the further increased dose, the modified dose for the subject is equal to the difference between the further increased dose and the incremental value for the last cycle repetition.

The amount of active ingredient that may be combined with the carrier materials to produce a single dosage form will vary depending upon the host treated and the mode of administration.

The compounds can be administered in various modes, e.g. orally, topically, or by injection. The precise amount of compound administered to a patient will be the responsibility of the attendant physician. In certain embodiments, the specific dose level for any patient will depend upon a variety of factors including the activity of the specific compound employed, the age, body weight, general health, sex, diets, time of administration, route of administration, rate of excretion, drug combination, the precise disorder being treated, and the severity of the indication or condition being treated. Also, the route of administration may vary depending on the condition and its severity.

In any case, multiple therapeutic agents (at least one of which is a compound disclosed herein) may be administered in any order or even simultaneously. If simultaneously, the multiple therapeutic agents may be provided in a single, unified form, or in multiple forms (by way of example only, either as a single pill or as two separate pills). One of the therapeutic agents may be given in multiple doses, or both may be given as multiple doses. If not simultaneous, the timing between the multiple doses may be any duration of time ranging from a few min to four weeks.

In certain embodiments, the disease chosen from primary hyperkalemic periodic paralysis, primary hypokalemic periodic paralysis, and related variants; Aland Island eye disease atrial fibrillation, Brugada syndrome, cardiomyopathy, cerebellar syndrome, cone-rod dystrophy, cystoid macular edema of retinitis pigmentosa, Dravet syndrome, epilepsy, epileptic encephalopathy, episodic ataxia, myokymia syndrome, episodic pain syndrome, hemiplegic migraine, febrile seizures, heart block, intracranial hypertension, long QT syndrome, neuropathy, night blindness, paroxysmal exercise-induced dyskinesia, Rett syndrome, sick sinus syndrome, spinocerebellar ataxia, sudden infant death syndrome (SIDS), Timothy syndrome, ventricular fibrillation, and paroxysmal kinesigenic choreoathetosis.

In certain embodiments, the disease chosen from primary hyperkalemic periodic paralysis, primary hypokalemic periodic paralysis, and related variants. In certain embodiments, the disease primary hyperkalemic periodic paralysis. In certain embodiments, the disease is primary hypokalemic periodic paralysis. In certain embodiments, the disease is a related variant to primary hyperkalemic periodic paralysis. In certain embodiments, the disease is a related variant to primary hypokalemic periodic paralysis.

In certain embodiments, the disease is Aland Island eye disease.

In certain embodiments, the disease is atrial fibrillation, such as familial atrial fibrillation.

In certain embodiments, the disease is Brugada syndrome, such as type 1 or type 3.

In certain embodiments, the disease is cardiomyopathy, such as dilated cardiomyopathy.

In certain embodiments, the disease is cerebellar syndrome in phosphomannomutase 2 (PMM2) deficiency, a congenital disorder of glycosylation.

In certain embodiments, the disease is cone-rod dystrophy, such as X-linked cone-rod dystrophy.

In certain embodiments, the disease is cystoid macular edema of retinitis pigmentosa.

In certain embodiments, the disease is Dravet syndrome.

In certain embodiments, the disease is epilepsy, such as generalized epilepsy, epilepsy type two, or epilepsy with febrile seizures.

In certain embodiments, the disease is epileptic encephalopathy, early infantile epileptic encephalopathy, which is an autosomal dominant form of the disease.

In certain embodiments, the disease is episodic ataxia, such as type 1, type 2, or type 5, or myokymia syndrome

In certain embodiments, the disease is episodic pain syndrome, such as familial episodic pain syndrome.

In certain embodiments, the disease is hemiplegic migraine types, familial hemiplegic migraine types 1 and 3.

In certain embodiments, the disease is febrile seizures, such as familial febrile seizures.

In certain embodiments, the disease is heart block, such as nonprogressive heart block, and progressive heart block type IA.

In certain embodiments, the disease is intracranial hypertension, such as idiopathic intracranial hypertension.

In certain embodiments, the disease is long QT syndrome-3

In certain embodiments, the disease is neuropathy, hereditary neuropathy, sensory neuropathy, and autonomic neuropathy type VII.

In certain embodiments, the disease is night blindness, such as congenital stationary night blindness, and X-linked night blindness.

In certain embodiments, the disease is paroxysmal exercise-induced dyskinesia.

In certain embodiments, the disease is Rett syndrome.

In certain embodiments, the disease is sick sinus syndrome.

In certain embodiments, the disease is spinocerebellar ataxia, such as spinocerebellar ataxia type 6.

In certain embodiments, the disease is sudden infant death syndrome (SIDS).

In certain embodiments, the disease is Timothy syndrome.

In certain embodiments, the disease is ventricular fibrillation, such as familial ventricular fibrillation.

In certain embodiments, the disease is paroxysmal kinesigenic choreoathetosis.

The human organic anion and cation transporters are classified within two Solute Carrier (SLC) superfamilies. The Solute Carrier Organic Anion (SLCO, formerly SLC21A) superfamily consists of organic anion transporting polypeptides (OATPs), while the organic anion transporters (OATs) and the organic cation transporters (OCTs) are classified in the solute carrier family 22A (SLC22A) superfamily. Individual members of each superfamily are expressed in epithelia throughout the body, where they absorb, distribute and eliminate drugs. Substrates of OATPs are large hydrophobic organic anions, while OATs transport smaller and more hydrophilic organic anions and OCTs transport organic cations. In addition to endogenous substrates, such as steroids, hormones and neurotransmitters, these proteins transport numerous drugs and other xenobiotics are transported, including statins, antivirals, antibiotics and anticancer drugs.

Expression of OATPs, OATs and OCTs can be regulated at the protein or transcriptional level and varies within each family by protein and tissue type. All three superfamilies consist of 12 transmembrane domain proteins with intracellular termini. Although no crystal structures have yet been determined, homology modelling and mutation experiments have explored the mechanism of substrate recognition and transport. Several polymorphisms identified in superfamily members have been shown to affect pharmacokinetics of their drug substrates, confirming the importance of these drug transporters for efficient pharmacological therapy.

An organic-anion-transporting polypeptide (OATP) is a membrane transport protein or “transporter” that mediates the transport of mainly organic anions across the cell membrane. Therefore, OATPs are the gatekeepers in the lipid bilayer of the cell membrane. OATP1B1, OATP1B3 and OCT1 are expressed on the sinusoidal membrane of hepatocytes and aid the accumulation of endogenous and xenobiotic compounds into hepatocytes for further metabolism or excretion into the bile. As well as expression in the liver, OATPs are expressed in many other tissues on basolateral and apical membranes, transporting anions, neutral and cationic compounds. They transport an extremely diverse range of drug compounds, including anticancer, antibiotic, lipid lowering drugs, anti-diabetic drugs, toxins and poisons.

Organic anion transporters (OATs in humans, Oats in rodents) are another family of multispecific transporters and are encoded by the SLC22/Slc22 gene superfamily. They mediate the transport of a diverse range of low molecular weight substrates including steroid hormone conjugates, biogenic amines, various drugs and toxins.

The organic anion transporter 1 (OAT1, solute carrier family 22 member 6, SLC22A6) is a protein that in humans is encoded by the SLC22A6 gene. It is a member of the organic anion transporter (OAT) family of proteins. OAT1 is a transmembrane protein expressed in the brain, placenta, eyes, smooth muscles, and basolateral membrane of proximal tubular cells of the kidneys. Along with OAT3, OAT1 mediates the uptake of a wide range of relatively small and hydrophilic organic anions from plasma into the cytoplasm of the proximal tubular cells of the kidneys. From there, these substrates are transported into the lumen of the nephrons of the kidneys for excretion.

Dicarboxylates, such as α-ketoglutarate generated within the cell or recycled from the extracellular space, are exchange substrates that fuel the influx of organic anions against their concentration gradient. When the uptake of one molecule of an organic anion is transported into a cell by an OAT1 exchanger, one molecule of an endogenous dicarboxylic acid (such as glutarate, ketoglutarate, etc.) is simultaneously transported out of the cell. Because endogenous dicarboxylic acid is constantly removed, OAT1 (OAT3)-positive cells risk depleting their supply of dicarboxylates. Once the supply of dicarboxylates is depleted, the OAT1 transporter can no longer function.

In certain embodiments, the OAT1 inhibitor is chosen from furosemide, diclofenac, naproxen, bumetanide, captopril, candesartan, losartan, chlorothiazide, cimetidine, ranitidine, telmisartan, olmesartan, simvastatin, fluvastatin, cefaclor, methotrexate, cefadroxil, cefoperazone, ceftizoxime, piperacillin, tazobactam, sulbactam, zidovudine, adefovir, cidofovir, ketorolac, diflunisal, and any combination thereof.

In certain embodiments, the OAT1 inhibitor is chosen from furosemide, diclofenac, naproxen, bumetanide, and any combination thereof. Furosemide and bumetanide are frequently used to prophylactically treat hyperkalemic periodic paralysis and to acutely treat muscle paralysis or myotonia. Diclofenac and naproxen, among other NSAIDs, are used frequently to manage muscle aches that result from attacks and myotonia, as well as bruises from falls that can occur in PPP.

In certain embodiments, the OAT1 inhibitor is chosen from telmisartan, ketorolac, diflunisal, and any combination thereof.

In certain embodiments, the OAT1 inhibitor is furosemide. In certain embodiments, the OAT1 inhibitor is diclofenac. In certain embodiments, the OAT1 inhibitor is naproxen. In certain embodiments, the OAT1 inhibitor is bumetanide. In certain embodiments, the OAT1 inhibitor is captopril. In certain embodiments, the OAT1 inhibitor is candesartan. In certain embodiments, the OAT1 inhibitor is losartan. In certain embodiments, the OAT1 inhibitor is chlorothiazide. In certain embodiments, the OAT1 inhibitor is cimetidine. In certain embodiments, the OAT1 inhibitor is ranitidine. In certain embodiments, the OAT1 inhibitor is telmisartan. In certain embodiments, the OAT1 inhibitor is olmesartan. In certain embodiments, the OAT1 inhibitor is simvastatin. In certain embodiments, the OAT1 inhibitor is fluvastatin. In certain embodiments, the OAT1 inhibitor is cefaclor. In certain embodiments, the OAT1 inhibitor is methotrexate. In certain embodiments, the OAT1 inhibitor is efadroxil. In certain embodiments, the OAT1 inhibitor is cefoperazone. In certain embodiments, the OAT1 inhibitor is ceftizoxime. In certain embodiments, the OAT1 inhibitor is piperacillin. In certain embodiments, the OAT1 inhibitor is tazobactam. In certain embodiments, the OAT1 inhibitor is sulbactam. In certain embodiments, the OAT1 inhibitor is zidovudine. In certain embodiments, the OAT1 inhibitor is adefovir. In certain embodiments, the OAT1 inhibitor is cidofovir. In certain embodiments, the OAT1 inhibitor is ketorolac. In certain embodiments, the OAT1 inhibitor is diflunisal.

OAT3 (organic anion transporter 3, solute carrier family 22 member 8, SLC22A8), is a protein that in humans is encoded by the SLC22A8 gene. Like OAT1, OAT3 transports and excretes organic anions. OAT3 depends indirectly on the inward sodium gradient and drives the reentry of dicarboxylates into the cytosol. The encoded protein is an integral membrane protein localized to the basolateral membrane of renal proximal tubule cells.

In certain embodiments, the OAT3 inhibitor is chosen from acyclovir, allopurinol, alprostadil, aminohippuric acid, avibactam, baricitinib, bumetanide, captopril, cefacetrile, cefaclor, cefazoline, cefdinir, cefotiam, ceftibuten, ceftizoxime, cephalexin, cephaloridine, cholic acid, cimetidine, ciprofloxacin, cyclic adenosine monophosphate (cAMP), diflunisal, dinoprostone, edaravore, ellagic acid, eluxadoline, estradiol, estrone, famotidine, fexofenadine, fluorescein, furosemide, glutaric acid, hydrocortisone, indomethacin, ketorolac, L-carnitine, L-citrulline, leucovorin, mercaptopurine, methotrexate, oseltamivir, oxalic acid, penicillin G (benzylpenicillin), piperacillin, pravastatin, quinapril, ranitidine, rosuvastatin, saxagliptin, silibinin A, sitagliptin, succinic acid, taurocholic acid, tazobactam, tenofovir, tetracycline, trifluridine, valaciclovir, valproic acid, and zidovudine.

In certain embodiments, the OAT3 inhibitor is chosen from telmisartan, ketorolac, diflunisal, and any combination thereof.

In some embodiments, the OAT3 inhibitor is acyclovir. In some embodiments, the OAT3 inhibitor is allopurinol. In some embodiments, the OAT3 inhibitor is alprostadil. In some embodiments, the OAT3 inhibitor is aminohippuric acid. In some embodiments, the OAT3 inhibitor is avibactam. In some embodiments, the OAT3 inhibitor is baricitinib. In some embodiments, the OAT3 inhibitor is bumetanide. In some embodiments, the OAT3 inhibitor is captopril. In some embodiments, the OAT3 inhibitor is cefacetrile. In some embodiments, the OAT3 inhibitor is cefaclor. In some embodiments, the OAT3 inhibitor is cefazoline. In some embodiments, the OAT3 inhibitor is cefdinir. In some embodiments, the OAT3 inhibitor is cefotiam. In some embodiments, the OAT3 inhibitor is ceftibuten. In some embodiments, the OAT3 inhibitor is ceftizoxime. In some embodiments, the OAT3 inhibitor is cephalexin. In some embodiments, the OAT3 inhibitor is cephaloridine. In some embodiments, the OAT3 inhibitor is cholic acid. In some embodiments, the OAT3 inhibitor is cimetidine. In some embodiments, the OAT3 inhibitor is ciprofloxacin. In some embodiments, the OAT3 inhibitor is cyclic adenosine monophosphate (cAMP). In some embodiments, the OAT3 inhibitor is diflunisal. In some embodiments, the OAT3 inhibitor is dinoprostone. In some embodiments, the OAT3 inhibitor is edaravore. In some embodiments, the OAT3 inhibitor is ellagic acid. In some embodiments, the OAT3 inhibitor is eluxadoline. In some embodiments, the OAT3 inhibitor is estradiol. In some embodiments, the OAT3 inhibitor is estrone. In some embodiments, the OAT3 inhibitor is famotidine. In some embodiments, the OAT3 inhibitor is fexofenadine. In some embodiments, the OAT3 inhibitor is fluorescein. In some embodiments, the OAT3 inhibitor is furosemide. In some embodiments, the OAT3 inhibitor is glutaric acid. In some embodiments, the OAT3 inhibitor is hydrocortisone. In some embodiments, the OAT3 inhibitor is indomethacin. In some embodiments, the OAT3 inhibitor is ketorolac. In some embodiments, the OAT3 inhibitor is L-citrulline. In some embodiments, the OAT3 inhibitor is leucovorin. In some embodiments, the OAT3 inhibitor is mercaptopurine. In some embodiments, the OAT3 inhibitor is methotrexate. In some embodiments, the OAT3 inhibitor is oseltamivir. In some embodiments, the OAT3 inhibitor is oxalic acid. In some embodiments, the OAT3 inhibitor is penicillin G (benzylpenicillin). In some embodiments, the OAT3 inhibitor is piperacillin. In some embodiments, the OAT3 inhibitor is pravastatin. In some embodiments, the OAT3 inhibitor is quinapril. In some embodiments, the OAT3 inhibitor is ranitidine. In some embodiments, the OAT3 inhibitor is rosuvastatin. In some embodiments, the OAT3 inhibitor is saxagliptin. In some embodiments, the OAT3 inhibitor is silibinin A. In some embodiments, the OAT3 inhibitor is sitagliptin. In some embodiments, the OAT3 inhibitor is succinic acid. In some embodiments, the OAT3 inhibitor is taurocholic acid. In some embodiments, the OAT3 inhibitor is tazobactam. In some embodiments, the OAT3 inhibitor is tenofovir. In some embodiments, the OAT3 inhibitor is tetracycline. In some embodiments, the OAT3 inhibitor is trifluridine. In some embodiments, the OAT3 inhibitor is valaciclovir. In some embodiments, the OAT3 inhibitor is valproic acid. In some embodiments, the OAT3 inhibitor is zidovudine.

In certain embodiments, the method further comprises informing the subject or a medical care worker that co-administration of dichlorphenamide, or a pharmaceutically acceptable salt thereof, and the OAT1 and/or OAT3 inhibitor may result in increased exposure of dichlorphenamide, or a pharmaceutically acceptable salt thereof.

In certain embodiments, the method further comprises informing the subject or a medical care worker that co-administration of dichlorphenamide, or a pharmaceutically acceptable salt thereof, and the OAT1 and/or OAT3 inhibitor may result in increased risk of one or more exposure-related adverse reactions associated with the dichlorphenamide, or a pharmaceutically acceptable salt thereof.

In certain embodiments, monitoring for signs and/or symptoms of toxicity comprises monitoring the serum concentration of the dichlorphenamide, or a pharmaceutically acceptable salt thereof.

In certain embodiments, the method further comprises monitoring the subject for one or more exposure-related adverse reactions. In certain embodiments, the one or more exposure-related adverse reactions are chosen from paresthesia, cognitive disorder, dysgeusia, confusional state, hypersensitivity reactions, anaphylaxis reactions, hypokalemia, metabolic acidosis, falls, amnesia, cardiac failure, condition aggravated, convulsion, fetal death, hallucination, nephrolithiasis, pancytopenia, psychotic disorder, renal tubular necrosis, stupor, syncope, and tremor.

In certain embodiments, the one or more exposure-related adverse reactions are chosen from paresthesia, cognitive disorder, dysgeusia, and confusional state. In certain embodiments, the one or more exposure-related adverse reactions are chosen from hypersensitivity reactions, anaphylaxis reactions, hypokalemia, metabolic acidosis, and falls. In certain embodiments, the one or more exposure-related adverse reactions are chosen from amnesia, cardiac failure, condition aggravated, convulsion, fetal death, hallucination, nephrolithiasis, pancytopenia, psychotic disorder, renal tubular necrosis, stupor, syncope, and tremor. In some embodiments, the one or more exposure-related adverse reactions is paresthesia. In some embodiments, the one or more exposure-related adverse reactions is cognitive disorder. In some embodiments, the one or more exposure-related adverse reactions is dysgeusia. In some embodiments, the one or more exposure-related adverse reactions is confusional state. In some embodiments, the one or more exposure-related adverse reactions is hypersensitivity reactions. In some embodiments, the one or more exposure-related adverse reactions is anaphylaxis reactions. In some embodiments, the one or more exposure-related adverse reactions is hypokalemia. In some embodiments, the one or more exposure-related adverse reactions is metabolic acidosis. In some embodiments, the one or more exposure-related adverse reactions is falls. In some embodiments, the one or more exposure-related adverse reactions is amnesia. In some embodiments, the one or more exposure-related adverse reactions is cardiac failure. In some embodiments, the one or more exposure-related adverse reactions is condition aggravated. In some embodiments, the one or more exposure-related adverse reactions is convulsion. In some embodiments, the one or more exposure-related adverse reactions is fetal death. In some embodiments, the one or more exposure-related adverse reactions is hallucination. In some embodiments, the one or more exposure-related adverse reactions is nephrolithiasis. In some embodiments, the one or more exposure-related adverse reactions is pancytopenia. In some embodiments, the one or more exposure-related adverse reactions is psychotic disorder. In some embodiments, the one or more exposure-related adverse reactions is renal tubular necrosis. In some embodiments, the one or more exposure-related adverse reactions is stupor. In some embodiments, the one or more exposure-related adverse reactions is syncope. In some embodiments, the one or more exposure-related adverse reactions is tremor.

In certain embodiments, adjusting the therapeutically effective amount of the dichlorphenamide, or a pharmaceutically acceptable salt thereof, comprises reducing the amount of the dichlorphenamide, or a pharmaceutically acceptable salt thereof, being administered.

In certain embodiments, the method further comprises reducing the dose and/or frequency of the dichlorphenamide, or a pharmaceutically acceptable salt thereof, administered to the subject based on the subject's ability to tolerate one or more exposure-related adverse reactions associated with the dichlorphenamide, or a pharmaceutically acceptable salt thereof. In certain embodiments, the dose of the dichlorphenamide, or a pharmaceutically acceptable salt thereof, is decreased. In certain embodiments, the dose of the dichlorphenamide, or a pharmaceutically acceptable salt thereof, is reduced by at least 5%, such as by at least 10%, by at least 20%, by at least 25%, by at least 30%, by at least 40%, by at least 50%, by at least 60%, by at least 70%, by at least 75%, by at least 80%, or by at least 90%. In certain embodiments, the frequency of administration of the dichlorphenamide, or a pharmaceutically acceptable salt thereof, is decreased. For example, when the dose is not reduced, the frequency of administration might be extended from twice daily (BID) to once daily (QD), or to every other day (QOD), and on. In certain embodiments, the method further comprises discontinuing administration of the dichlorphenamide, or a pharmaceutically acceptable salt thereof, based on the patient's ability to tolerate one or more exposure-related adverse reactions.

Besides being useful for human treatment, certain compounds and formulations disclosed herein may also be useful for veterinary treating companion animals, exotic animals and farm animals, including mammals, rodents, and the like. More animals include horses, dogs, and cats.

Examples of embodiments of the present disclosure are provided in the following examples. The following examples are presented only by way of illustration and to assist one of ordinary skill in using the disclosure. The examples are not intended in any way to otherwise limit the scope of the disclosure.

### Example

A study was designed to evaluate dichlorphenamide as a substrate of human transporters (including, OAT1 and OAT3). Compounds that are substrates of the transporters may be victims or perpetrators in drug-drug interactions. Experiments were carried out as described in the FDA and EMA draft guidance documents for Drug Interaction Studies (FDA 2017, EMA 2013).

Human embryonic kidney 293 (HEK293) cells expressing transporter transfected with vectors containing human transporter cDNA for OAT1 and control cells (HEK293 cells transfected with only vector) were used in experiments to evaluate dichlorphenamide as an inhibitor of OAT1.

Dichlorphenamide was prepared in dimethyl sulfoxide (DMSO) and spiked into incubation media for a final concentration of 0.1% v/v DMSO. HEK293 cells were cultured in DMEM supplemented with FBS (8.9% v/v), antibiotic/antimycotic (0.89% v/v) and L-glutamine (1.79 mM). The medium was replaced every 1 to 3 days, and the cells were passaged when confluent. Cells were cultured on a 24-well tissue plate.

Non-specific binding of the test articles to the incubation vessels without cells was evaluated by incubating dichlorphenamide in incubation media at low and high concentrations (1 and 1000 μM for dichlorphenamide) in 24-well plates or a 24-well transwell plate at 37±2° C. for either 30 or 120 min. At the end of the incubation period, aliquots of the mixtures were collected, analyzed by LC MS/MS and compared to the dose solutions (100% solution).

Probe substrates and positive control inhibitors were prepared in DMSO at a concentration 1000-fold higher than the incubation concentration and spiked into incubation medium each at 0.1% v/v DMSO. The final concentration of DMSO was 0.2% v/v and was equal in all incubations (e.g., the sum of the DMSO from the probe substrate and dichlorphenamide, positive control inhibitor or the solvent control [DMSO]). The final concentration of DMSO was 0.1% v/v in no solvent control incubations.

Probenecid (Probalan™) increases uric acid excretion in the urine for treating gout and hyperuricemia. Probenecid interferes with the kidneys' OATs, which reclaims uric acid from the urine and returns it to the plasma. Probenecid has drug-drug interactions with captopril, indomethacin, ketoprofen, ketorolac, naproxen, cephalosporins, quinolones, penicillins, methotrexate, zidovudine, ganciclovir, lorazepam, and acyclovir. In all these interactions, the excretion of these drugs is reduced due to probenecid.

p-Aminohippurate (p-aminohippuric acid, PAH, PAHA) is the glycine amide of p-aminobenzoic acid. It is filtered by the glomeruli and is actively secreted by the proximal tubules. At low plasma concentrations (1.0 to 2.0 mg/100 mL), an average of 90% of aminohippurate is cleared by the kidneys from the renal blood stream in a single circulation.

Estrone-3-sulfate (estrone sulfate, E1S) is a natural, endogenous steroid and an estrogen ester and conjugate. E1S itself is biologically inactive, with less than 1% of the relative binding affinity of estradiol for the ERα and ERβ, but it can be converted by steroid sulfatase (also called estrogen sulfatase) into estrone, which is an estrogen. Exogenous estrogens are metabolized in the same manner as endogenous estrogens. Circulating estrogens exist in a dynamic equilibrium of metabolic interconversions which occur mainly in the liver.

After cell culture, culture medium was removed, and incubation medium was added to the cells. After about 10 min, transepithelial/transendothelial electric resistance (TEER) was recorded to determine cytotoxicity and cells were preincubated at 37±2° C. for 30 to 60 min. After preincubation, incubation medium with probe substrate containing the solvent control, control inhibitor, dichlorphenamide was added to the donor chamber and incubation medium containing the solvent control, control inhibitors, dichlorphenamide was added to the receiver chamber for total incubation volumes of 200 and 980 μL for the apical and basolateral chambers, respectively. Samples (100 μL) were collected from the receiver compartment at 120 min. In wells in which the recovery was calculated, samples (20 μL) were taken from the donor chambers at the start of the incubation (time zero) and after the final time point (120 min). If the donor chamber was sampled at time zero, the volume added to the donor chamber at time zero was 20 μL higher (220 or 1000 μL). Samples containing the probe substrate were mixed with internal standard and analyzed by LC MS/MS.

The ability of dichlorphenamide to inhibit the accumulation of probe substrates into transporter-expressing and control cells was measured to evaluate dichlorphenamide as inhibitors of SLC transporters. Inhibition of transporters was determined by incubating the cells with a probe substrate and dichlorphenamide and measuring the amount of probe substrate accumulating in the cells.

Radiolabeled substrates were dried under a stream of nitrogen then reconstituted in non-labeled substrate or solvent. Probe substrates and positive control inhibitors were prepared in DMSO at a concentration 1000-fold higher than the incubation concentration and spiked into incubation medium each at 0.1% v/v DMSO. The final concentration of DMSO was 0.2% v/v and was equal in all incubations. That is, the sum of the DMSO from the probe substrate and dichlorphenamide, positive control inhibitor or the solvent control (DMSO) were equal. The final concentration of DMSO was 0.1% v/v in no solvent control incubations. Incubations of HEK293 cells were performed in HBSS buffer containing sodium bicarbonate (4 mM) and HEPES (9 mM), pH 7.4.

After incubation, incubation medium was removed, and cells were rinsed once with 1 mL of ice-cold phosphate-buffered saline (PBS) containing 0.2% w/v bovine specific antigen (BSA) and twice with ice-cold PBS. The PBS was removed, and 0.5 mL of sodium hydroxide (0.1 M) was added and pipetted up and down to dissolve and suspend the cells. An aliquot of the medium was added to a 96 well plate, diluted with scintillation fluid and analyzed on a MicroBeta2 scintillation counter. The amount of protein in each incubation was determined by bicinchoninic acid (BCA) analysis.

Tables 1 and 2 show that dichlorphenamide is a substrate of OAT1. Tables 3 and 4 show that dichlorphenamide is a substrate of OAT3. Where applicable, n is the number of replicates, NA is Not applicable, and SD refers to the standard deviation. Unless otherwise noted, values are triplicate determinations rounded to three significant figures with standard deviations rounded to the same degree of accuracy. Percentages are rounded to one decimal place except percentages ≥100, rounded to the nearest whole number.

TABLE 1
OAT1 substrate determination in HEK293 cells

| | | Uptake (pmol/mg protein) | | | | |
|---|---|---|---|---|---|---|
| Substrate | Incubation | (Average ± SD) | | Uptake | Recovery (%) | |
| concentration (μM) | time (min) | Control | OAT1 | ratio | Control | OAT1 |
| Dichlorphenamide | 1 | 45.4 ± 0.4 | 109 ± 2 | 2.41 | 95.3 | 98.5 |
| (10 μM) | 3 | 123 ± 6 | 294 ± 14 | 2.39 | | |
| | 10 | 265 ± 8 | 591 ± 11 | 2.23 | | |
| Dichlorphenamide | 1 | 42.2 ± 2.4 | 54.4 ± 2.7 | 1.29 | NA | NA |
| (10 μM) (+) | 3 | 121 ± 8 | 135 ± 6 | 1.11 | | |
| Probenecid | 10 | 284 ± 19 | 286 ± 17 | 1.01 | | |
| (100 μM) | | | | | | |

TABLE 2
OAT1 substrate determination in HEK293 cells using [³H]-p-Aminohippurate
(1 μM) as the probe substrate (positive-control inhibition)

| | Uptake (pmol/mg protein) | | Background corrected | |
|---|---|---|---|---|
| | (Average ± SD) | | uptake rate | Percent of |
| Inhibitor | Control | OAT1 | (pmol/mg/min) | control |
| Solvent control | 1.33 (n = 2) | 17.0 ± 1.6 | 15.7 | 100 |
| Probenecid (100 μM) | 0.487 ± 0.070 | 1.43 ± 0.73 | 0.940 | 6.0 |

TABLE 3
Dichlorphenamide: OAT3 substrate determination in HEK293 cells

| | | Uptake (pmol/mg protein) | | | | |
|---|---|---|---|---|---|---|
| Substrate | Incubation | (Average ± SD) | | Uptake | Recovery (%) | |
| concentration (μM) | time (min) | Control | OAT3 | ratio | Control | OAT3 |
| Dichlorphenamide | 1 | 147 ± 47 | 333 ± 24 | 2.26 | 68.8 | 77.9 |
| (10 μM) | 3 | 118 ± 3 | 311 ± 29 | 2.64 | | |
| | 10 | 169 ± 9 | 463 ± 27 | 2.75 | | |
| Dichlorphenamide | 1 | 124 ± 13 | 123 ± 7 | 0.987 | NA | NA |
| (10 μM) (+) | 3 | 125 ± 5 | 126 ± 18 | 1.00 | | |
| Probenecid | 10 | 143 ± 41 | 177 ± 16 | 1.24 | | |
| (100 μM) | | | | | | |

TABLE 4
OAT3 substrate determination in HEK293 cells using [³H]-Estrone-3-sulfate
(50 nM) as the probe substrate (positive-control inhibition)

| | Uptake (pmol/mg protein) | | Background corrected | |
|---|---|---|---|---|
| | (Average ± SD) | | uptake rate | Percent of |
| Inhibitor | Control | OAT3 | (pmol/mg/min) | control |
| Solvent control | 0.0770 ± 0.0140 | 1.25 ± 0.02 | 0.585 | 100 |
| Probenecid (100 μM) | 0.0670 ± 0.0124 | 0.104 ± 0.006 | 0.0184 | 3.2 |

The various embodiments described above can be combined to provide further embodiments. All the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims but should be construed to include all possible embodiments along with the full scope of equivalents to which such claims are entitled. Accordingly, the claims are not limited by the disclosure.

1.-29. (canceled)

30. A method of treating a disease chosen from primary hyperkalemic periodic paralysis, primary hypokalemic periodic paralysis, and related variants in a subject in need thereof,
wherein the subject is exhibiting one or more signs of dichlorphenamide toxicity as a result of increase in exposure of dichlorphenamide caused by the subject being co-administered:
(i) an organic anion transporter-1 (OAT1) substrate dichlorphenamide, or a pharmaceutically acceptable salt thereof, at an initial dosage of up to 200 mg daily to treat a disease chosen from primary hyperkalemic periodic paralysis, primary hypokalemic periodic paralysis, and related variants, and
(ii) an organic anion transporter-1 (OAT1) inhibitor to treat a disease other than primary hyperkalemic periodic paralysis, primary hypokalemic periodic paralysis, and related variants, wherein the OAT1 inhibitor is chosen from furosemide, diclofenac, naproxen, bumetanide, captopril, candesartan, losartan, chlorothiazide, cimetidine, ranitidine, telmisartan, olmesartan, simvastatin, fluvastatin, cefaclor, methotrexate, cefadroxil, cefoperazone, ceftizoxime, piperacillin, tazobactam, sulbactam, zidovudine, adefovir, cidofovir, ketorolac, diflunisal, and any combination thereof,
wherein the one or more signs of dichlorphenamide toxicity is chosen from paresthesia, cognitive disorder, dysgeusia, confusional state, hypersensitivity reactions, anaphylaxis reactions, hypokalemia, metabolic acidosis, falls, amnesia, cardiac failure, condition aggravated, convulsion, fetal death, hallucination, nephrolithiasis, pancytopenia, psychotic disorder, renal tubular necrosis, stupor, syncope, and tremor,
the method comprising:
administering a reduced dose of dichlorphenamide, or a pharmaceutically acceptable salt thereof, once daily to the subject, wherein the reduced dose is less than the initial dose; and
continuing administration of the OAT1 inhibitor.

31. A method of treating a disease chosen from primary hyperkalemic periodic paralysis, primary hypokalemic periodic paralysis, and related variants in a subject in need thereof,
wherein the subject is also in need of treatment with an organic anion transporter-1 (OAT1) inhibitor to treat a disease other than primary hyperkalemic periodic paralysis, primary hypokalemic periodic paralysis, and related variants, and
wherein the OAT1 inhibitor is chosen from furosemide, diclofenac, naproxen, bumetanide, captopril, candesartan, losartan, chlorothiazide, cimetidine, ranitidine, telmisartan, olmesartan, simvastatin, fluvastatin, cefaclor, methotrexate, cefadroxil, cefoperazone, ceftizoxime, piperacillin, tazobactam, sulbactam, zidovudine, adefovir, cidofovir, ketorolac, diflunisal, and any combination thereof,
the method comprising:
administering the OAT1 inhibitor to the subject; and
subsequently administering dichlorphenamide, or a pharmaceutically acceptable salt thereof, to the subject at a reduced dose to compensate for the expected increase in exposure resulting from co-administration of the OAT1 inhibitor and the OAT1 substrate dichlorphenamide, or a pharmaceutically acceptable salt thereof,
wherein the reduced dose is relative to what the subject would be administered if the subject was not being administered the OAT1 inhibitor.
